(19) United States
(12) Patent Application Publication
(10) Pub. No.: US 2010/0106193 A1
(43) Pub. Date: Apr. 29, 2010

(54) SYSTEM AND METHOD FOR ALIGNING VERTEBRAE IN THE AMELIORATION OF ABERRANT SPINAL COLUMN DEVIATION CONDITIONS IN PATIENTS REQUIRING THE ACCOMODATION OF SPINAL COLUMN GROWTH OR ELONGATION

(76) Inventor: Mark A. Barry, (US)

Correspondence Address: DAVID G. HENRY;Patton Boggs LLP, 2001 Ross Avenue, Suite 3000, Dallas, TX, 75021, US

(21) Appl. No.: 12/258,488

(22) Filed: Oct. 27, 2008

(51) Int. Cl. A61B 17/70; A61B 17/86; A61B 17/88

(52) U.S. Cl. 606/264; 606/261; 606/305; 606/279

(57) ABSTRACT

A system and method for ameliorating spinal column anomalies, such as scoliosis, while accommodating growth of juvenile patients, includes pedicle screws and a spinal rod of non-circular cross section. Each pedicle screw includes spinal rod engagement mean for achieving a slide-only engagement between each segment of said spinal rod that is respectively engaged with the pedicle screw. Pedicle screws are thereby allowed to slide longitudinally along the spinal rod as attached vertebrae move during growth, while movement in other directions is arrested in order to preserve a proper orientation of involved vertebrae to maintain scoliotic correction in three dimensions.

### BACKGROUND OF THE INVENTION

1. Field of The Invention

The present invention relates to methods and apparatus for management and correction of spinal deformities, such as scoliosis.

2. Background Information

A serious deficiency presently exists with respect to conventional treatment and instrumentation for treating spinal deviation anomalies (such as scoliosis). This is particularly true as relates to juvenile cases involving greater than 45° curvatures (as such terminology is understood in the field) and more particularly to idiopathic scoliosis.

Currently, idiopathic scoliosis (“I.S.”) comprises approximately 75% of all juvenile cases. Those I.S. cases involving curvatures in the 25°-45° range indicate treatment through bracing (beginning roughly at the bottom end of this range), but become untreatable by bracing roughly at the top end of this range). Curvatures in excess of 45° indicate surgical intervention.

Use of implanted spinal rod systems of the current art introduce significant patient risks. These include considerable likelihood of hardware dislodgement (such as when hooks are used to engage spinal rod system components), ulcerations of skin that overlies protrusions of implanted systems, premature fusion of adjacent vertebrae with highly deleterious growth and spinal contour issues, impairment of longitudinal spinal growth, worsening of axial plane deformities such as rib hump, and aggravation of truncal balance problems.

To make matters worse, existing rod spinal rod systems, when used in juveniles, require periodic lengthening to accommodate growth (roughly every 9-18 months). Further still, the existing systems only control curvature in two dimensions. Finally, a formal fusion procedure is required at or near skeletal maturity.

An ideal system for addressing the present shortcomings of treatment options for juvenile scoliosis involving greater than 45° curvatures is one which (at least): (1) provides three-dimensional correction of spinal anomalies; (2) provides secure engagement between instrumentation of affected vertebrae; (3) obviates the need for periodic lengthening procedures; and (4) obviates the need for formal fusions at skeletal maturity.

Such a system would only be possible were it to “grow” with the patient, utilize other than easily dislodgeable skeletal engagement means, and maintain desired orientation and alignment of vertebrae in all dimensions.

With respect to this latter objective: current spinal rods are of circular cross section. Were attachment means for present spinal rods to be left “loose” to accommodate longitudinal motions along the rods as vertebrae move relatively as a result of growth, there would be nothing to combat the axial rotation of the vertebrae (relative the spinal rod) even as they are constrained in their longitudinal movement along the rod. Such axial rotation would result in far less than optimal correction of the overall spinal topography.

Were an ideal system for addressing juvenile scoliosis victims requiring surgical intervention to become available (addressing each of the above-listed shortcomings of the systems and methods of the present art), the recipients would benefit in at least the following ways: (1) they would enjoy a much higher incidence and degree of success in alleviating their spinal deformities (in all dimensions of spinal column topography); (2) they would achieve more nearly normal growth expectations; (3) they would be spared from multiple surgical procedures with their associated risks; (4) they would not face the painful and potentially catastrophic consequences of spinal rod system component dislodgement; and (5) they would maintain mobility at adulthood that would otherwise be lost though otherwise required fusions.

### SUMMARY OF THE INVENTION

In view of the foregoing, it is an object of the present invention to provide an improved system of spinal instrumentation for use in ameliorating aberrant spinal column deviation conditions, such as scoliosis, particularly (though not necessarily solely) in juvenile cases of idiopathic scoliosis.

It is another object of the present invention to provide an improved system and associated method for ameliorating aberrant spinal column deviation conditions, such as scoliosis, which system and method addresses each of the above-listed shortcomings of the spinal rod systems and methods for addressing juvenile scoliosis that is of the present art.

It is another object of the present invention to provide an improved system and associated method for ameliorating aberrant spinal column deviation conditions, such as scoliosis, which system and method reduce hazards to patients relating at least to implantation of instrumentation, subsequent post-implantation surgical interventions related to accommodation of patient growth, spontaneous vertebral fusions, and inhibition of normal growth of the spine.

It is another object of the present invention to provide an improved method for ameliorating aberrant spinal column deviation conditions, such as scoliosis, which system accommodates growth without surgical intervention to the degree required of spinal rod systems of the present art.

It is another object of the present invention to provide an improved system of spinal instrumentation, and a method for the use thereof, for ameliorating aberrant spinal column deviation conditions, such as scoliosis, which system and method facilitate maintaining spinal correction in three dimensions, rather than the mere two dimensions presently achievable (to a limited degree, and with limited success) with systems and methods of the present art.

In satisfaction of each of the stated objects, as well as objects of natural extension thereof, the inventor's present invention provides a system and method for use of such system which will afford its recipients at least the following benefits: (1) a much higher incidence and degree of success in alleviating their spinal deformities (in all dimensions of spinal column topography); (2) achievement of more nearly normal growth expectations; (3) the avoidance of multiple surgical procedures and associated discomfort and risks otherwise required in association with presently available spinal rod systems; (4) the elimination of substantially all risk spinal rod system component dislodgement; and (5) the maintenance of mobility at adulthood that would otherwise be lost though otherwise required fusions.

The spinal rod system of the present invention, the method for use of which is intended primarily to treat cases of juvenile scoliosis involving curvatures of greater than 45°, includes, in summary, pedicle screws with spinal rod engagement means for slidably engaging spinal rods of non-circular cross section to facilitate longitudinal, patient growth-related movement of the pedicle screws. Because of the complimentary contours of the non-circular spinal rods and engagement means of the associated, specially configured pedicle screws, a “slide-only engagement” is achieved. That is to day that longitudinal movement of the pedicle screws is allowed, while at the same time axial rotation and other undesirable movement of the pedicle screw heads relative to the rod (and the relative motion of the attached vertebrae) is nearly, completely arrested. Therefore, once the spinal rod is itself contoured according to the desired spinal topography, optimal scoliotic correction (in three dimensions) is achieved, not only at the time of initial implantation, but is perpetuated as the patient grows.

Optimal methods for achieving the initial scoliotic correction in three dimensions, which the present invention will maintain for the growing (juvenile) patient are best illustrated through reference to U.S. Patent Application, Publication No. 20060195092, which Application (and resulting Patent, if any) is hereby incorporated by reference.

### BRIEF DESCRIPTION OF THE DRAWINGS

The present invention may be more easily understood with reference to figures, which are as follow:

FIG. 1 is a diagrammatic, dorsal view of a spinal column with a growing spinal rod system of the present invention attached to selected vertebrae thereof.

FIG. 2 is a perspective depiction of an example of a pedicle screw having the unique spinal rod engagement means of the present invention for slidably engaging the spinal rod (shown in FIGS. 1 and 4) to permit longitudinal motion of the pedicle screw relative to the spinal rod, while preventing axial rotation of the screw (and associated vertebrae) relative to the spinal rod.

FIG. 3 is a diagrammatic side elevational view of a spinal column having the preferred three pedicle screw “clusters” situated for engaging a spinal rod for practice of the method of the present invention.

FIG. 4 is a diagrammatic, perspective view of two adjacent pedicle screws of the present invention, shown engaged with a non-circular cross sectional spinal rod of the same in the “slide-only engagment” that is achievable, and is an object of the present invention.

### DETAILED DESCRIPTION OF THE PREFERRED EMBODIMENT

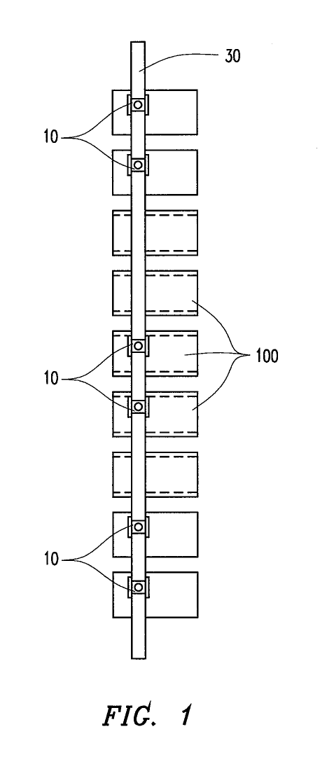

With reference to FIGS. 1-4 the growing rod spinal deviation correction system of the present invention includes a number of pedicle screws 10, each implanted in respective vertebrae 100 to which forces will be applied by way of a properly contoured spinal rod 30, initially to achieve a scoliotic correction in an initial surgical intervention, and thereafter to maintain the desired correction, even as the patient grows.

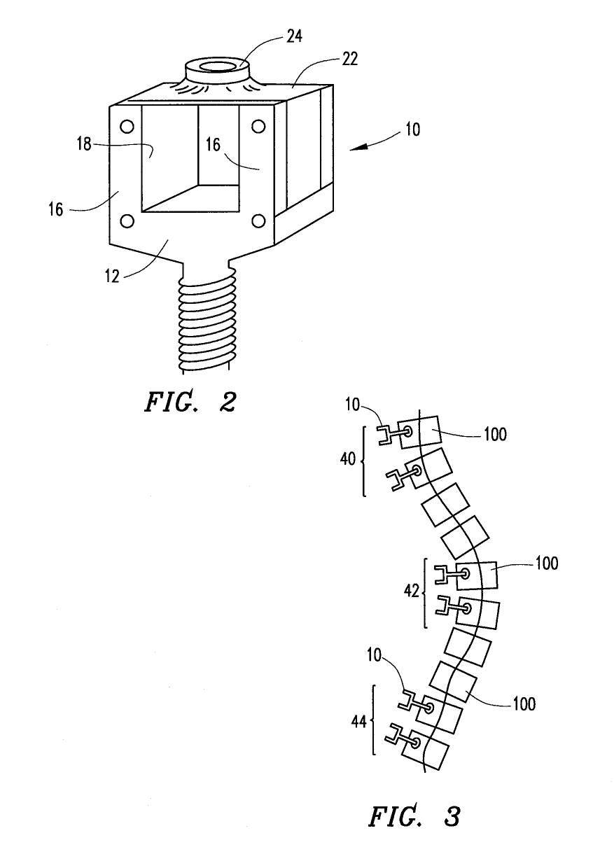

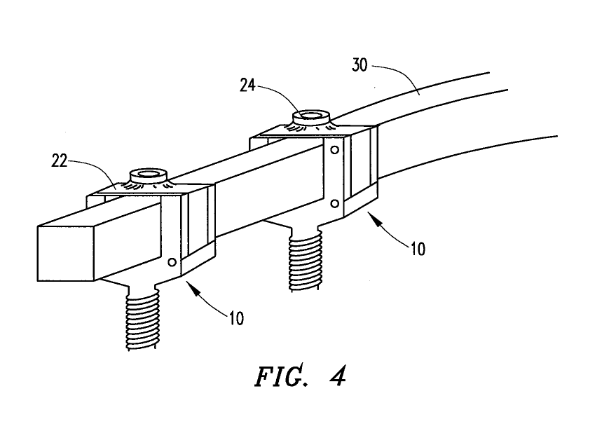

With particular reference to FIGS. 2 and 4, pedicle screws 10 and spinal rod 30 are respectively configured such that pedicle screws 10 may, in a “slide-only engagement” slide longitudinally along the length of spinal rod 30, but the same are constrained from any axial rotation and other undesirable movement because of the respective geometry of the spinal rod 30 and the portion of pedicle screws 10 with which the spinal rod 30 is mechanically linked (the “rod engagement means”).

The depicted embodiment of spinal rod 30 shown in the drawings is of a substantially square cross sectional geometry, and the associated spinal rod engagement means is configured in a complimentary fashion for both: (1) allowing longitudinal movement of the pedicle screws 10 relative to spinal rod 30 and (2) preventing axial rotation and other undesirable movement of the pedicle screw 10 relative to spinal rod 30. However, it must be understood that other “non-circular” geometries for spinal rod 30 and the rod engagement means of pedicle screws 10 may be substituted for that shown herein as a preferred embodiment. For example cross sectional geometries (“non-circular geometries”) for spinal rods 30 may include (among others not listed) those which are triangular, hexagonal, rectangular, gear-toothed, cross-shaped, or ovoid, with the spinal rod engagement means portion of pedicle screws 10 being of a complimentary geometry. In each such case, by virtue of the relatively tight, nested engagement between a spinal rod 30 of non-circular cross sectional geometry with a spinal rod engagement means portion of pedicle screws 10 of a complimentary geometry, substantially no axial rotation of pedicle screw 10 relative to spinal rod 30 is possible, and the engagement between pedicle screws 10 and spinal rod 30 is a “slide-only engagement.”

In the preferred embodiment of the pedicle screws 10 of the present invention, the head portion 12 of pedicle screws 10 is configured as a yoke-like structure. Two, upwardly projecting arms 16 cooperatively form this structure, defining a rod enclosure space 18, itself having a lateral opening 20 through which a segment of spinal rod 30 may be laterally introduced into the rod enclosure space 18.

A snap-fit clip 22 serves to occlude opening 20 and thereby constrain the associated length of spinal rod 30 within space 18. A set screw 24 is provided for clip 22 for use in instances where longitudinal movement of pedicle screws 10 relative to spinal rod 30 is to be prevented (to be discussed hereafter). In such cases, set screw 24 is adjusted in such a manner that it engages the adjacent surface of spinal rod 30 whereby substantially all relative movement between spinal rod 30 and pedicle screw 10 is arrested.

Referring particularly to FIGS. 1 and 3, the preferred method for use of the present growing rod system involves, by way of an example involving a right thoracic curve, placing pedicle screws 10 in three clusters. An upper cluster 40 involves two pedicle screws 10 placed in vertebrae 100 above the upper end vertebrae (“UEV” in FIG. 3) of the scoliotic curve; a middle cluster 42 placed in vertebrae 100 substantially at the apex of the scoliotic curve; and a lower cluster 44 placed in vertebrae 100 below the lower end vertebrae (“LEV” in FIG. 3) of the scoliotic curve.

Once spinal rod 30 is engaged with pedicle screws 10, and the initial three-dimensional scoliotic correction is achieved, and once clips 22 are engaged with each of the pedicle screws 10, set screws 24 of pedicle screws 10 of the middle cluster 42 are tightened to “anchor” spinal rod 30, while set screws 24 (if any) of the remaining clips 22 are left disengaged to allow the earlier-described longitudinal movement of those latter pedicle screws 10 along spinal rod 30.

Once the present spinal rod system is implanted, as described, a juvenile patient's subsequent growth is unhindered by the system, while correction of the scoliotic curve is maintained to maturity and thereafter. Proper relative alignment of the vertebrae is maintained, as is the individual orientation of affected vertebrae, thereby achieving and maintaining a true three-dimensional scoliotic correction.

Although the invention has been described with reference to specific embodiments, this description is not meant to be construed in a limited sense. Various modifications of the disclosed embodiments, as well as alternative embodiments of the inventions will become apparent to persons skilled in the art upon reference to the description of the invention. It is, therefore, contemplated that the appended claims will cover such modifications that fall within the scope of the invention.

I claim:

1. An improved spinal rod system comprising:
a spinal rod having a non-circular cross sectional geometry; and
a plurality of pedicle screws, each said pedicle screw having spinal rod engagement means for achieving a slide-only engagement between a segment of said spinal rod and said pedicle screw for allowing longitudinal movement of said pedicle screw relative to said spinal rod, while resisting axial rotation of said pedicle screw relative to said spinal rod; and
spinal rod securing mean configured for interfacing with said pedicle screw and thereafter for securing an mechanical engagement between a said segment of said spinal and said spinal rod engagement means.

2. The system of claim 1 wherein said spinal rod engagement means comprises constituents of a head portion of said pedicle screw which define a spinal rod enclosure space which is contoured in such a manner as to positively engage a segment of said spinal rod in a manner for substantially preventing relative movement between said pedicle screw and said spinal rod in all directions other than substantially along the longitudinal axis of said spinal rod.

3. The system of claim 2 wherein said spinal rod exhibits a non-circular cross sectional geometry, and said spinal rod engagement means of each said pedicle screw defines said rod enclosure space of a complimentary geometry for achieving said slide-only engagement between said segment of said spinal rod and a plurality of said pedicle screws.

4. A method for correcting and maintaining correction of a scoliotic curvature of the spine comprising the steps of:
selecting a growing rod spinal rod system, itself comprising:
a spinal rod having a non-circular cross sectional geometry; and
a plurality of pedicle screws, each said pedicle screw having spinal rod engagement means for engaging a segment of said spinal rod in such a slide-only manner for allowing longitudinal movement of said pedicle screw relative to said spinal rod, while resisting axial rotation of said pedicle screw relative to said spinal rod; and
spinal rod securing means configured for interfacing with said pedicle screw and thereafter for securing an mechanical engagement between a said segment of said spinal and said spinal rod engagement means;
implanting a first said pedicle screw in an upper vertebrae;
implanting a second pedicle screw in a middle vertebrae;
implanting a third pedicle screw in a lower vertebrae;
aligning a plurality of vertebrae to achieve a scoliotic correction;
engaging said spinal rod with said spinal rod engagement means respectively of said first, second and third pedicle screws; and
engaging said spinal rod securing means to each of said first, second and third pedicle screws.

5. The method of claim 4 wherein said spinal rod engagement means comprises constituents of a head portion of said pedicle screw which define a spinal rod enclosure space which is contoured in such a manner as to positively engage a segment of said spinal rod in a manner for substantially preventing relative movement between said pedicle screw and said spinal rod in all directions other than substantially along the longitudinal axis of said spinal rod.

6. The system of claim 5 wherein said spinal rod exhibits a non-circular cross sectional geometry, and said spinal rod engagement means of each said pedicle screw defines said rod enclosure space of a complimentary geometry for achieving said slide-only engagement between said segment of said spinal rod and a plurality of said pedicle screws.
